(19) United States
(12) Patent Application Publication
(10) Pub. No.: US 2006/0025755 A1
(43) Pub. Date: Feb. 2, 2006

(54) Surgical cooling system and method

(76) Inventors: Jaime Landman, New York, NY (US); Caroline Dorothea Ames, Bloomington, IN (US); Stephen Thomas Dryer, Cincinnati, OH (US); Salvatore P. Sutera, Clayton, MO (US); Tabitha Yun-Ching Shen, Walnut Creek, CA (US); Kelley Virginia Foyil, Tampa, FL (US)

Correspondence Address: HARNESS, DICKEY, & PIERCE, P.L.C, 7700 BONHOMME, STE 400, ST. LOUIS, MO, 63105, US

(21) Appl. No.: 11/193,551

(22) Filed: Jul. 29, 2005

Related U.S. Application Data
(60) Provisional application No. 60/592,790, filed on Jul. 30, 2004.

(51) Int. Cl. A61B 18/18

(52) U.S. Cl. 606/20

(57) ABSTRACT

The present invention provides a system for cooling a body part during surgery that includes a surgical cooling solution generator for producing a surgical cooling solution such as an ice slush from a working solution, a surgical cooling solution transport system for transporting the surgical cooling solution, an applicator configured for receiving the surgical cooling solution from the supply line and having a first mode and a second mode for respectively routing the surgical cooling solution to the recycle line for recycling, and for routing the surgical cooling solution to a surgical delivery interface. The applicator comprises an intake port for receiving the surgical cooling solution, a recycle port for transmitting a portion of the surgical cooling solution for recycling, and a surgical delivery interface for transmitting a portion of the surgical cooling solution to a body cavity. A method is also provided for transmitting a continuous flow of the surgical cooling solution to an applicator, selectively delivering a portion of the continuous surgical cooling solution flow to the body cavity, and recycling the non-delivered portion of the surgical cooling solution flow for inclusion in transmitting a continuous flow of the surgical cooling solution to the applicator.

### CROSS-REFERENCE TO RELATED APPLICATIONS

This application claims the benefit of U.S. Provisional Application No. 60/592,790, filed on Jul. 30, 2004. The disclosure of the above application is incorporated herein by reference.

### FIELD OF THE INVENTION

The present invention relates to a cooling system and method of use during surgery and, more specifically, relates to a system and method of use for cooling an internal organ or tissue before and during surgery such as laparoscopy.

### BACKGROUND OF THE INVENTION

Surgeons utilize minimally invasive laparoscopy during surgery to reduce the impacts of surgery on the patient. For example, urologic surgeons utilize partial nephrectomies in order to conserve maximal renal function in cases of renal cell carcinoma (RCC) and other renal cancers. In these surgeries, only the tumor or cancerous portion of the kidney is removed, as compared to the entire kidney in a radical nephrectomy. If the tumor is less than 4 cm in diameter, laparoscopic surgery is chosen as a minimally invasive alternative to open surgery. In this laparoscopic procedure, three small incisions, ranging from 5 to 10 mm in length, replace the one 20-50 cm incision used in traditional nephrectomies. One of these incisions is used for the videoscope and the other two are used for laparoscopic surgical instruments. After the incisions are made, trocars are inserted through the incisions and into the abdomen. The trocars have airtight one-way valves and are configured for receiving a videoscope and the surgical instruments. The abdominal cavity is inflated with carbon dioxide gas to allow the surgeons more room to work and to better view the kidney. The images from the videoscope are viewed by the surgeon on a monitor positioned near the patient.

An interruption of the blood supply to any tissue in the body results in tissue ischemia. This condition is often required in order that surgeons may operate in a bloodless field. During nephrectomies, the renal artery and renal vein are clamped at the start of the procedure to prevent excessive bleeding during surgery. Warm ischemia is accomplished at normal body temperature of 37° C., and the allowable ischemic time for a kidney at this temperature is approximately 30 minutes. After 30 minutes, the kidney suffers irreversible damage (necrosis) and post-operative kidney function is significantly reduced. During partial nephrectomies, the diseased tissue is removed and sent to a pathologist for testing and for reporting back to the surgeon while the patient is still on the operating table. If the pathologist determines that more tissue should be removed from the patient, the entire surgery can take more than the 30-minute window allowed by warm ischemia. In this case, the surgeon must resort to a traditional radical nephrectomy since the kidney function will begin to deteriorate after 30 minutes. In contrast, cold ischemia can significantly increase the operating-time window required for these complex surgeries, possibly by as much as 3 hours.

Cold ischemia is accomplished by reducing the temperature of the organ before and during surgery. In cold ischemia, the organ should be cooled to about 15° to reduce the metabolism and thereby the oxygen requirements of the organ. These lower temperatures provide for dramatically increased ischemic time since at these temperatures the organs, such as kidneys, do not need as much oxygen from the blood. Considerable research has been devoted to cold ischemia, and it has been shown that when ischemia is induced at lower temperatures, the post-operative tissue is also much healthier.

In the field of laparoscopy, there have been several attempts at achieving the desired cold ischemia. Existing solutions include arterial perfusion, intra-renal cooling (circulation of ice-cold saline through the kidney), hand-assist laparoscopy, and use of an endoscopic ice bag. When carried out properly and flawlessly, these methods have produced moderate cooling effects. However, problems and risks have occurred that have challenged their effective utilization for general use in the operating room.

Arterial perfusion cools the kidney through the kidney's own vasculature. The renal artery is perfused with a cold saline solution having an approximate temperature of −1.7° C. Because the renal artery is punctured in order to perfuse the artery, the integrity of the artery is compromised and causes additional problems and patient risks. Furthermore, there is a risk of volume overload to the artery and/or organ, i.e., perfusing too much saline into the artery and overloading the vasculature. There is also a risk of inducing systemic hypothermia with this approach.

Intra-renal perfusion is similar to arterial perfusion. The renal artery is clamped and cold saline is irrigated through the ureter into the kidney's collecting ducts. However, this method of cooling does not bring the kidney to a uniform temperature and tissue temperatures may vary by as much as 6° C. across the kidney. Intra-renal perfusion can also cause major discomfort in the patient, as a catheter must remain in the patient for several days postoperatively to drain the saline. The intrusion into the collecting duct system also poses additional risks of high pressure and damage to the ducts.

Hand-assisted laparoscopy is the most invasive of the current techniques to cool the kidney to achieve cold ischemia. In this procedure, an incision is made in the abdomen that is large enough to admit the surgeon's hand. Once the incision is made, a surgical glove filled with ice slush is packed around the kidney. This method effectively decreases the temperature of the kidney to as low as 5° C. However, the ice filled glove must be removed to clear the operating field. Subsequently, therefore, the kidney will gradually warm up until its average temperature is about 19° C., which is slightly greater than the optimal temperature for cold ischemia. Another disadvantage to this procedure is the larger incision required, such that the procedure is no longer minimally invasive and incurs many of the disadvantages of traditional surgeries. Additionally, this method generally allows only one surgical instrument to be used at a time.

Still another method uses an endoscopic bag to encapsulate the kidney with ice to achieve renal hypothermia. To allow this technique, the kidney must first be completely dissected from its surrounding connective tissue in order to allow the endoscopic bag to be placed around the kidney. The surgeon fills the bag with ice-slush from a syringe through the trocar. Afterward, the bag is retrieved through a 12 mm hole. This method is technically difficult and may take as long as 20 minutes to setup and deploy. Once the kidney reaches hypothermia, the surgeon must perform the cumbersome and time consuming task of dissecting and removing the endoscopic bag in order to operate on the kidney.

The above-described methods and devices are problematic and present limitations to the application of effective cold ischemia during surgery.

### SUMMARY OF THE INVENTION

Understanding the limitations of the pre-existing systems, the inventors have developed, in one aspect, a system for cooling body organs and tissues during surgery. The system includes a surgical cooling solution generator for producing a surgical cooling solution from a working solution, with the produced surgical cooling solution having substantial consistency. The system can be deployed through a small caliber end-effector. The system can also include a surgical cooling solution transport system for transporting the surgical cooling solution. The transport system includes a supply line and a recycle line. The system can further include an applicator with a surgical delivery interface. The applicator is configured for receiving the surgical cooling solution from the supply line. The applicator has a first mode for routing the surgical cooling solution to the recycle line for recycling of the surgical cooling solution (that can minimize substantial melting of the ice slush) and a second mode for routing the surgical cooling solution to a surgical delivery interface for delivery about a body part.

Another aspect of the invention is an applicator for dispensing a surgical cooling solution during surgery. The applicator includes an intake port for receiving the surgical cooling solution and a recycle port for transmitting a portion of the surgical cooling solution for recycling. The applicator also includes a surgical delivery interface for transmitting a portion of the surgical cooling solution, such as an ice slush, to a body cavity. The applicator further includes a flow control system for controlling a flow of the received surgical cooling solution. The flow control system has a first mode for routing the surgical cooling solution to the recycle port and a second mode for routing the surgical cooling solution to the surgical delivery interface.

In yet another aspect, the invention is a method of providing a surgical cooling solution to a body cavity during surgery. The method includes generating a surgical cooling solution having a predetermined consistency. The predetermined surgical cooling solution can be an ice slush having an appropriate consistency for deployment through a small caliber end-effector. The method also includes transmitting a continuous flow of the surgical cooling solution to an applicator and selectively delivering a portion of the continuous surgical cooling solution flow to the body cavity. The method further includes recycling the non-delivered portion of the continuous surgical cooling solution flow for inclusion in said transmitted continuous flow of the surgical cooling solution to the applicator.

In still another aspect, the present invention is a method of cooling tissue during laparoscopic surgery, including transmitting a continuous flow of the surgical cooling solution, such as a fine consistency ice slush, to a laparoscopic surgical cooling solution applicator. The method also includes selectively delivering a portion of the continuous surgical cooling solution flow to a body cavity through a trocar and recycling the non-delivered portion of the continuous flow of the surgical cooling solution for inclusion in the continuous flow of the surgical cooling solution through the laparoscopic applicator.

Further aspects of the present invention will be in part apparent and in part pointed out below. It should be understood that various aspects of the invention can be implemented individually or in combination with one another. It should also be understood that the detailed description and drawings, while indicating certain exemplary embodiments of the invention, are intended for purposes of illustration only and should not be construed as limiting the scope of the invention.

### BRIEF DESCRIPTION OF THE DRAWINGS

The present invention will become more fully understood from the detailed description and the accompanying drawings.

FIG. 1 is a schematic diagram of a surgical cooling system for cooling a body part during surgery according to one exemplary embodiment of the invention.

FIG. 2 is a detailed front perspective view of a cooling solution generator according to one embodiment of the invention.

FIGS. 3A and 3B are side perspective views and exploded views, respectively, of an applicator for applying a cooling solution, such as a fine consistency ice slush, during surgery according to one exemplary embodiment of the invention.

FIGS. 4A to 4E are various views of a second applicator for a cooling solution, such as a fine consistency ice solution, according to another exemplary embodiment of the invention.

FIG. 5 is a side sectional view of the internal parts of a tubing pinch device for a flow control system of an applicator according to one exemplary embodiment of the invention.

FIG. 6 is a graph illustrating temperature versus time data from a test of one exemplary embodiment of a surgical cooling system.

Corresponding reference characters indicate corresponding parts throughout the several views of the drawings.

### DETAILED DESCRIPTION OF EXEMPLARY EMBODIMENTS

The following description is merely exemplary in nature and is in no way intended to limit the invention, its applications, or uses.

In some embodiments, the invention includes a system for cooling a body part/organ or tissue during surgery wherein the system has a surgical cooling solution generator for producing a surgical cooling solution from a working solution. The surgical cooling solution can, for example, be a fine consistency ice slush. In one embodiment, the fine consistency ice slush is selected to enable delivery of the surgical cooling solution through a small caliber end-effector. The system also includes a cooling solution transport system for transporting the cooling solution, and an applicator is configured for receiving the cooling solution from the supply line. The applicator can have a first mode for routing the cooling solution to the recycle line for recycling, and a second mode for routing the cooling solution to a surgical delivery interface. The surgical cooling solution generator can produce a cooling solution having substantial consistency. The transport system can include a supply line and a recycle line through which the cooling solution is transported.

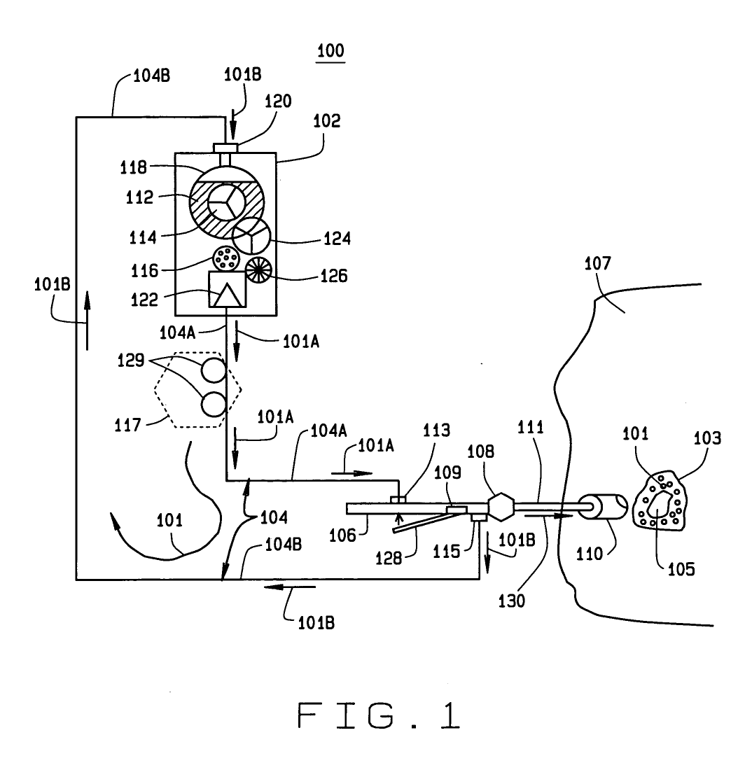

Referring to FIG. 1, one embodiment of the present invention comprises a system 100 for cooling a part or region in a subject body such as an organ or tissue surrounding or adjacent to an organ. The system 100 includes a surgical cooling solution generator 102 for producing a cooling solution 101, such as an ice slush, to be transported and delivered to a body cavity or body part prior to and during surgery. The cooling solution 101 is produced from a working solution 112 (such as an aqueous working solution), and has a substantial viscous consistency when produced. The cooling solution 101 can be a fine consistency ice slush of such viscosity and fine consistency as to enable delivery of the ice slush through a small caliber device such as an end-effector. A transport system 104 provides for the continuous flow of the input cooling solution 101A through the system 100 and to an applicator 106. The applicator 106 includes an intake port 113 for receiving the input cooling solution 101A and a surgical delivery interface 108 for routing the surgical cooling solution 130 to a surgical site such as a cavity 103 or an organ 105 of a patient body 107. In some embodiments, this includes an end effector 111 that is operable with a trocar 110 for use in laparoscopic surgery.

The applicator 106 also includes a recycle port 115 for diverting and/or providing the recycled cooling solution 101B to the recycle transport line or system 104B. An applicator control system 109, which can be a valve, controls the routing and flow of the cooling solution 101 within the applicator 106 from the intake port 113 to either the recycle port 115 or the surgical delivery interface 108. The applicator control system 109 can provide for 100 percent of the input cooling solution 101A entering the intake port 113 to be either routed to the recycle port 115 or the surgical delivery interface 108. Additionally, the applicator control system 109 can provide for routing of a variable portion of the input cooling solution 101A to be routed to the surgical delivery interface 108 with the remainder of the input cooling solution 101A being routed to the recycle port 115. The transport system 104 includes a delivery transport system 104A and a recycle transport system 104B that interoperate with the applicator 106 and the recycle port 120 of the surgical cooling solution generator 102 to provide for a circulation flow of the cooling solution 101 through the system 100.

One exemplary embodiment of the surgical cooling solution generator 102 as shown in FIG. 1 was implemented by the inventors by modifying an Ultra-2 Specialty Drink Machine manufactured by Bunn Commercial Equipment. The surgical cooling solution generator 102 can alternatively be modified or constructed from any other apparatus suitable for producing a comparable cooling solution 101, such as an ice slush, or can be entirely designed and constructed independent of a commercially available apparatus. The surgical cooling solution generator 102 can have one or two cooling hoppers 118, each hopper 118 having a rotating auger 114 that rotates so as to continuously mix the cooling solution 101 held in hopper 118. The surgical cooling solution generator 102 can include a stainless steel screening device 116 to screen out or otherwise filter or prevent large ice particles having a size greater then a predetermined dimension from entering the transport system 104. A second rotating auger 124 can also provide additional mixing or agitation and/or provide an extruding force to the cooling solution 101 for establishing a satisfactory consistency to the cooling solution 101. The first auger 114 and second auger 124 can both be used to mix and pump the cooling solution 101 to be delivered by the transport system 104. In some situations or embodiments, the mixing and pumping/cycling can be continuous or intermittent as required to ensure satisfactory operation of the system without clogging or freezing.

The surgical cooling solution generator 102 can be used to generate pressure in the transport system 104 in lieu of a separate pump 117, and could optionally drive the cooling solution 101 through a grinding device 126 that reduces the average granule dimension in the cooling solution 101 to ensure proper operation, e.g., prevent clogging. The surgical cooling solution generator 102 could therefore include a first rotating auger 114, a second rotating auger 124, and a grinding device 126, where first auger 114 is configured for continuously mixing the cooling solution 101 held within the hopper 118 and the second auger 124 pumps a cooling solution flow through the grinding device 126. Additionally, an end adaptor 122 can provide for connecting one or more tubes or other components to receive the cooling solution 101 from the hopper 118.

One or more hoppers 118 act as reservoirs for holding the working solution 112 and producing the cooling solution 101. The cooling solution 101 is mixed and recycled through hoppers 118 to keep the transport system 104 primed with the cooling solution 101 having a consistent temperature or within a specified temperature range. In some embodiments, such mixing and/or pumping or recycling can be continuous to ensure the desired consistency of the cooling solution 101 during surgery. In one embodiment, as described above, the surgical cooling solution generator 102 can include two separate hoppers 118 (not shown), each equipped with an individual cooling drum. The surgical cooling solution generator 102 can be capable of freezing various amounts of working solution 112 into cooling solution 101 (for example between 10-15 liters or in another example about 13 liters) in each hopper 118 simultaneously to that of operating room consistency in about three hours. Once the cooling solution 101 is generated from the working solution 112, the cooling solution 101 remains in hoppers 118 and is mixed/agitated by rotating augers 114 to prevent coalescence of the ice slush granules into large aggregates, e.g., prevent the formation of ice chunks within the cooling solution 101.

When the surgical cooling solution generator 102 is active in the recycle mode, a pump 117, or other flow control device such as one of the augers as described above, is not activated by the operator, e.g., such as the surgeon. In some embodiments as will be discussed below, the pump 117 is a pump having rollers 129 for non-invasively pumping the cooling solution 101. In this mode, the cooling solution 101 leaves the hopper 118, flows through the delivery transport system 104A, into the applicator 106, and returns to the hopper 118 via the recycle transport system 104B. The surgical cooling solution generator 102 re-cools the recycled cooling solution 101B and pumps it back through the transport system 104. The operation of the second mode whereby the cooling solution 101 is delivered by the applicator 106 to the end effector 111 as surgical cooling solution 130 will be as described below.

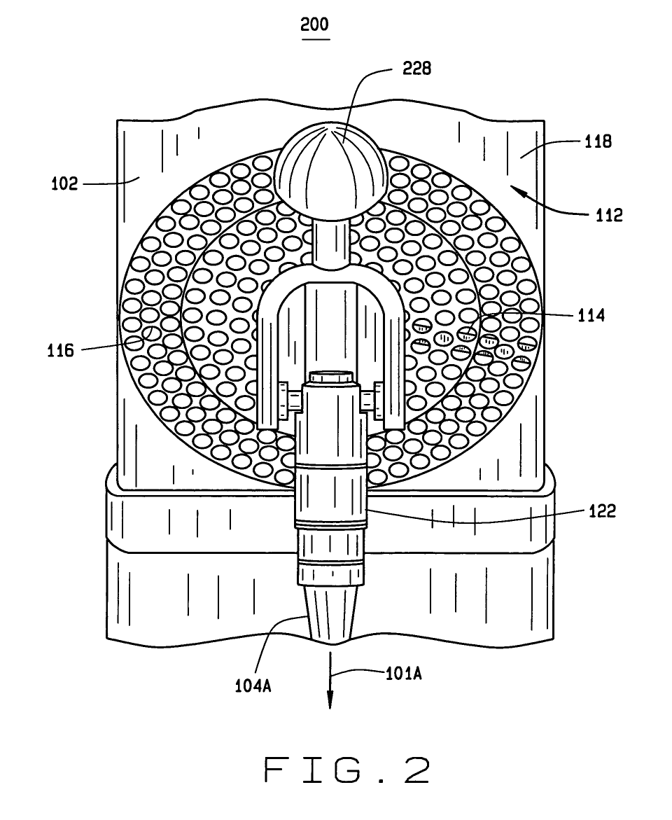

Referring now to FIG. 2, a generator 200 illustrates one exemplary portion of the surgical cooling solution generator 102 that includes the hopper 118, the auger 114, the screen filter 116, and an end adaptor 122. The end adaptor 122 adapts an output of the surgical cooling solution generator 102 to the delivery transport system 104A for transmitting or delivering the recycle cooling solution 101B to the applicator 106.

The surgical cooling solution generator 102 can further include a stainless steel slush delivery spout adaptor 122 for connecting the surgical cooling solution generator 102 to the transport system 104, and includes a standard o-ring to provide a seal with hopper 118. The adapter 122 is held in place with a screw positioned perpendicular from the side outflow valve.

The surgical cooling solution generator 102 should be sterile for surgical applications and should generate a consistent volumetric flow of the cooling solution 101. Further, it can be capable of controlling both the flow rate and the consistency of the cooling solution 101. Additionally, the surgical cooling solution generator 102 is capable of freezing a large volume of liquid into a cooling solution 101 having a controlled-consistency within a predetermined amount of time.

For example, in one implementation by the inventors, the surgical cooling solution generator 102 was designed to provide up to one liter per minute of the cooling solution 101 through the transport system 104, but could also have the capability to supply 25% less than this maximum flow rate. As discussed above, the modified Bunn Specialty Drink Maker, model CDS-2, produced more than six liters of a smooth, almost toothpaste-consistency saline ice slush cooling solution in one and a half hours and had a maximum production capacity of 13.25 liters (3.5 gallons) of the cooling solution 101 in three hours for one of the two available hoppers 118. This particular system required an electrical supply of 120 V AC, 15 amps, for a maximum power requirement of 180 watts. The surgical cooling solution generator 102 can use Freon or another refrigerant to cool a stainless steel cooling drum to freeze the working solution 112 such as a 0.9% saline solution. Inside the surgical cooling solution generator 102, the rotating auger 114 simultaneously mixes and pumps the contents (working solution 112 and/or cooling solution 101) of the hopper 118. The surgical cooling solution generator 102 and the system 100 are well suited for an operating room environment.

The working solution 112 in the surgical cooling solution generator 102 can be a saline solution having about a 0.9 percent saline concentration, which the surgical cooling solution generator 102 freezes into slush of a near toothpaste consistency. In some embodiments, the saline solution is a sterile solution. The working solution 112 and/or cooling solution 101 can be comprised of any solution suitable for application during surgery. In another embodiment, the working solution 112 is a readily-available 0.9% saline (9 g NaCl/L deionized water) slush solution. However, other cooling solutions 101 and/or working solutions 112 can also be used and be consistent with the scope of the system and method of the invention. The selection of the working solution 112 and the cooling solution 101 can be based on various factors including the salinity of the solution and the osmotic or other impact that such a solution can have on any body organ or tissue that will be exposed to the cooling solution 101. Other factors may include the amount of time required to prepare the cooling solution 101 from the working solution 112 to the appropriate consistency, the consistency required by the cooling solution 101, and the availability of the working solution 112.

The transport system 104 for transporting the cooling solution 101 includes a supply line of the delivery transport system 104A for delivering the input cooling solution 101A and a recycle line of the transport system 104B for transporting the recycled cooling solution 101B. Both the supply line of the delivery transport system 104A and recycle line of the transport system 104B are in communication with the applicator 106. The transport system 104 lines (104A and 104B) can be made of ½ inch flexible vinyl tubing or other suitable means for enabling delivery of the cooling solution 101. Such tubing can be fabricated from Tygon® tubing, which is a registered trademark of, and is manufactured by, Saint-Gobain Performance Plastics that is economical and disposable thereby mitigating cost and sterility concerns. In one embodiment, the tubing size is a 0.5 inch inner diameter tubing with 3/32 inch wall thickness (1.27 cm inner diameter tubing with 2.381 mm walls), which corresponds with SciLog's size 24. In another embodiment, to maximize the amount of slush flow, a larger diameter tube can be used over most of the tubing length and then stepped down to a 10mm inner diameter at the applicator. In one embodiment, a Tygon® tubing one-half inch (1.27 cm) inner diameter× 3/32 inch (2.38 mm) wall thickness, connected to a ⅜ inch (9.52 mm) inner diameter× 3/32 inch (2.38 mm) wall thickness tubing via a one-half inch to ⅜ inch disposable plastic reducing connector. This tubing provides ample flow rate with minimal ice clogging at the applicator 106.

The supply line and the recycle line of the delivery transport system 104A and 104B, respectively, can include tubing connectors (not shown) secured by clamps or cable ties, to secure the tubing to the applicator 106 or any other fitting in the transport system 104. This can include a generally available cable tie fastener.

The cooling solution 101 flows to the applicator 106 via the supply line of the delivery transport system 104A. When the input cooling solution 101A enters the applicator 106, the cooling solution 101A can be routed in two directions through a T-connector, valve or other controlling system. These two slush pathways are the recycled cooling solution 101B and the surgical cooling solution 130. Due to the mechanisms inside the applicator 106, only one path can be fully open at any given time; however, in some embodiments a variable division of the flow between the two paths can be provided.

The applicator 106 can include a surgical delivery interface 108 and can be configured to receive the input cooling solution 101A from the supply line of the delivery transport system 104A via the intake port 113. The applicator 106 has a first mode for routing the input cooling solution 101A to a recycle port 115 that connects to the recycle line of the transport system 104B for recycling the cooling solution 101 as the recycled cooling solution 101B (such as an ice slush). A second mode routes the input cooling solution 101A to a surgical delivery interface 108 as the surgical cooling solution 130. The applicator 106 can include a handle 228 operated manually by a user, and can provide the user control of the selection of the first mode or the second mode of operation. The applicator 106 can further include variable routing modes to provide a plurality of variable combinations of the first mode and the second mode, where the variable routing modes delivers variable routing of the input cooling solution 101A to surgical delivery interface 108 thereby producing the surgical cooling solution 130. The surgical delivery interface 108 can include an end effector 111 configured for operation with the laparoscopic trocar 110 inserted into a body 107 of the patient. The surgical cooling solution 130 is introduced through the trocar 110 and delivered into the cavity 103 of the body 107 and delivered (such as layering) around an organ 105, such as a kidney, a heart, etc.

The pump 117 can also generate a predetermined pressure in the supply or delivery portion of the transport system 102. The pump 117 can be any type of pump and in one embodiment is a roller pump with one or more rollers 129 that do not contact the sterile cooling solution 101. Such pump 117 can be used in lieu of the optional second auger 124 option described above. The pump 117 can optionally be incorporated to generate pressure in the transport system 104. The selection of pump 117 can be based on a number of characteristics. The pump 117 can include variable operating speeds which range from 59-2258 milliliter per minute, a plurality of rollers for pumping, a digital control, a self-priming feature with reversible flow, and a remote control run/stop capability. Additionally, as this can be used in an operating room environment, a small size and low power consumption is also desirable.

The pump 117 can also be manually operated or controlled by a computer (not shown) with a unidirectional or bi-directional control. In some embodiments, the pump 117 can be a roller pump such as a digital pump or a perfusion control system. In such an embodiment, the pump 117 can be fitted with a peristaltic pump head, which is intended for “thick-walled” tubing. This particular pump 117, while intended for use with Silicone, C-Flex or PharMed tubing, can also be used with Tygon® tubing. In order to achieve a one liter per minute flow rate, the ACCU CP-20 can be selected. However, other pumps 117 can be chosen to provide various other flow rates and pressure levels. Roller pumps use standard-sized tubing and never come into contact with the contents of the tubing. Therefore, roller pumps are suitable for use in some embodiments of the system 100. Roller pumps can also control the flow rate. Another example of the pump 117 is a peristaltic meter pump that can be practical and transportable, yet just as efficacious as a roller pump. Such a pump 117 applies a tangential occlusive force to the supply line of the delivery transport system 104A running from the surgical cooling solution generator 102 machine to the applicator 106. Applying a peristaltic motion, it pushes slush toward the applicator 106. Examples of roller pumps include the COBE perfusion system or the ACCU Scilog series meter. Pump speeds of 65 RPM have yielded good results in studies, but higher flow rates may be possible and desirable.

To prevent connection failure under the pressure applied by the pump 117, cable ties (not shown) can be used to secure a supply line of the delivery transport system 104A and a recycle line of the transport system 104B to tubing connectors. To reduce any pressure buildup due to changes in the tubing size, a ½ inch to ⅜ inch tubing converter can be placed inline near the surgical cooling solution generator 102 and before the tubing of the transport system 104 enters the pump 117. In this embodiment, with the exception of 0.25 m of ½ inch tubing connecting the surgical cooling solution generator 102 to the pump 117, the remaining 2 meters of tubing is ⅜ inch in diameter. The recycling of the cooling solution stream or flow prevents melting of the cooling solution 101 within the supply line of the delivery transport system 104A and recycle line of the transport system 104B, and thereby minimizes the amount of water or liquefying slush in the cooling solution 101 admitted to the surgical site.

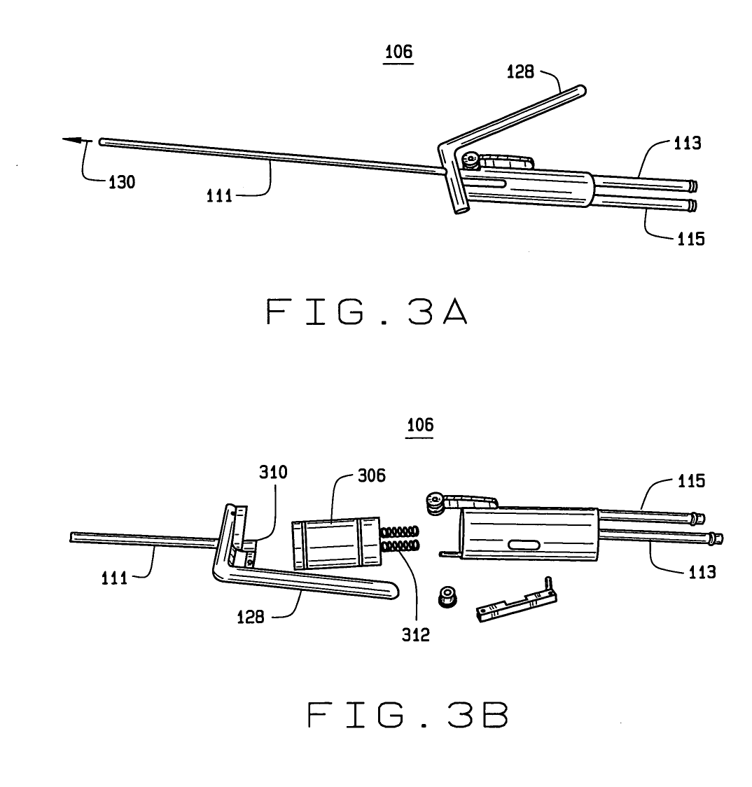

Another aspect of the present invention is the applicator 106 for dispensing the cooling solution 101 during surgery. As shown in FIG. 3a, the applicator 106 includes the intake port 113 for receiving the cooling solution 101A and the recycle port 115 for transmitting all, or a portion of, the cooling solution 101 as the recycled cooling solution 101B. The applicator 106 includes the end effector 111 transmitting the surgical cooling solution 130 of the cooling solution 101 before or during surgery. The applicator 106 includes a flow control system having a first and second mode for controlling the cooling solution 101 flow to the recycle port 115 and to the end effector 111, respectively.

For laparoscopic surgery, an end effector 111 can have a 10 millimeter dimension and be used with a standard 10-12 mm laparoscopic trocar 110 for insertion into the cavity 103 of the subject body 107, or about an organ 105 or surrounding tissue. The trocar 110 is a sharp-pointed surgical instrument, used with a cannula, to puncture a body cavity for fluid aspiration and is well known to one skilled in the art. The surgical cooling solution 130 is routed through the end effector 111, which can be aimed through a standard 10-12 mm laparoscopic trocar 110. The surgical cooling solution 130 can then be delivered to parenchyma or to an organ such as a kidney or heart.

The two modes of flow control system receive a user control for selecting the first mode and the second mode. In the first mode, the cooling solution 101A does not flow to the subject body, but flows into the recycle line of the transport system 104B and exits through the transport system 104 to be returned to the surgical cooling solution generator 102 as recycle solution 101B. The intake port 113 and the recycle port 115 of the applicator 106 are configured to be coupled to the delivery transport system 104A and the recycle transport system 104B, and their respective fluid lines. The cooling solution 101 constantly circulates through the transport system 104, the surgical cooling solution generator 102 and the applicator 106 thereby preventing melting or clogging of the cooling solution 101 or ice slush in the transport system 104. In the second mode, the handle 128 is depressed by a user and slush flows in and is directed to the end effector 111. Flow control system includes a valve 306 that is biased by a spring 310. When activated by the handle, the valve 306 withdraws under the pressure of the bias spring 312 from against surgical exit interface 310 allowing the surgical cooling solution 130 to flow to the end effector 111. The end effector 111 is configured for operation with the laparoscopic trocar 110 to provide delivery and distribution of the cooling solution 101 into the subject body. When the handle 128 is released by the user, the applicator 106 automatically reverts to the first mode.

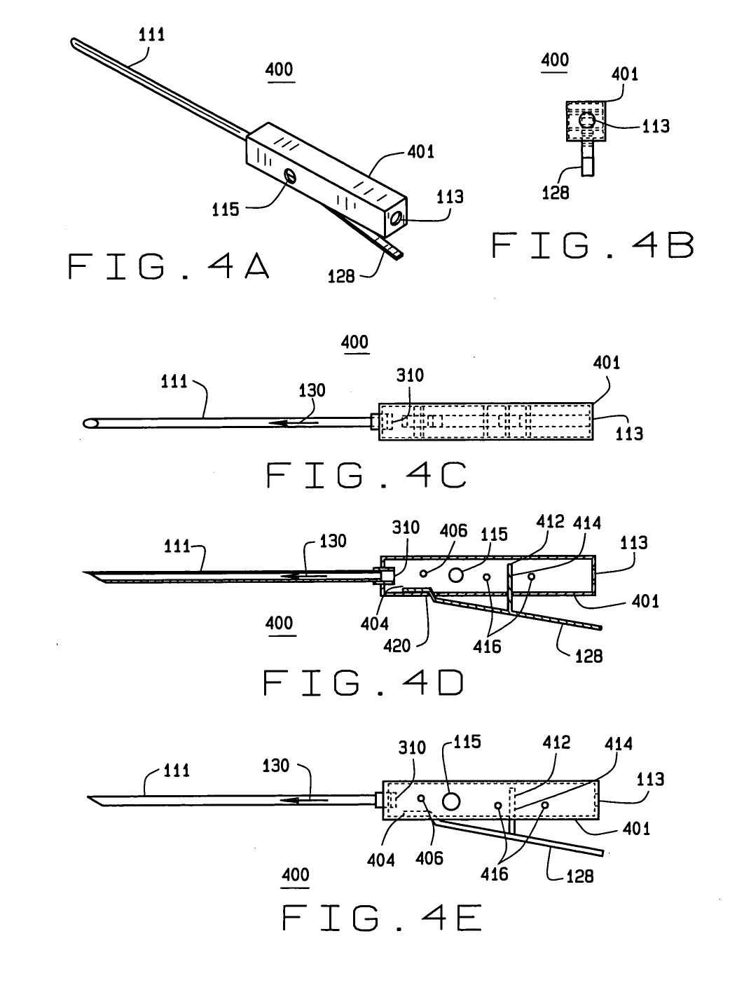

Referring now to FIGS. 4A-E and FIG. 5, the applicator 106 is illustrated with a tubing pinch control applicator 400. In this embodiment, a variable mode is provided for dividing the amount of the cooling solution 101 delivered or routed to the surgical delivery interface 108 as the surgical cooling solution 130. The tubing pinch control applicator 400 includes an applicator body 401 and the handle 128. A first occluding finger 404 and a second occluding finger 414 are coupled to the handle 126. The handle 128 operates on a fulcrum pin 420 to select between occlusion by first finger 404 or second finger 414. One or more occlusion pegs 406, 412, and 416 on the applicator body 401 interoperate with the first and second occluding fingers 404 and 414, respectively, to occlude tubing containing the cooling solution 101.

The applicators such as those shown, by way of example, as the tubing pinch control applicator 400 and the applicator 106, are designed to enable the surgeon to control the flow rate and placement of the cooling solution 101 by manipulating a handle 128 and/or a control system that occludes or pinches one or more flexible tubes. The flow rate is controlled by changing the occlusion of the surgical cooling solution 130 and the recycled cooling solution 101B; the greater the fractional occlusion of a particular segment of tubing and the lower the flow rate passing through that segment. To prevent the surgical cooling solution 130 and the recycled cooling solution 101B from being occluded at the same time, the occluding fingers 404 and 414 shown in FIG. 4D occlude the tubing by moving in a teeter-totter-like fashion; as one finger pinches a tube segment, the other finger withdraws from the other tube, thereby relieving its occlusion. The handle 128 of the applicator 106 can be the portion of the handle 128 that directly moves in a teeter-totter-like fashion, or its action can be indirect. Thus, the user control of the first and second modes or the variable mode providing a variable flow of the cooling solution 101 is operable by the hand of the user through the handle 128. The variable mode routes a user-defined portion of the cooling solution 101 to the surgical delivery interface 108.

The handle 128 is operated by a user and can include a variable mode for routing a user-defined portion of the cooling solution 101 to the surgical delivery interface, where the handle 128 controls the selection of the first mode, the second mode, and the user-defined portion of the cooling solution 101 flow to be delivered to the surgical delivery interface 108. As the surgeon operates the handle 128, the occluding finger for the recycle stream moves up, forcing the occluding finger for the exit stream to move down. In the resting position, the default flow pathway is through the recycle stream.

A spring (not shown) is placed beneath the exit stream tubing, which is occluded by an occluding finger, held in place by spring. The spring exerts enough force (approximately 2.8N or 20 lb-ft) to keep the exit stream occluded until the spring is compressed by the surgeon. The recycled cooling solution 101B is not occluded in the default position, so it will take the recycle stream pathway back to the hopper 118 while the surgeon is not using the device. While the surgeon is operating the handle 128, the surgeon needs to overcome the forces exerted by the occluding fingers 404 and 414. The occluding finger 404 for the exit stream needs to resist the upwards force of the spring, which is 89.0N (20 lb), and the occluding finger 414 for the recycle stream will be pushed by the opening of the tubing, which will exert a force of 62.8N (14lb). In total, the surgeon will need to squeeze the handle 128 with a force of approximately 108N (24 lb). It should be noted that the flow control system utilizing the occluding fingers 404 and 414 and functioning as a tube pinching device can also be replaced by a flow control valve. The mechanism for flow control can also be in the form of a foot-pedal, a hand-trigger or a hand-squeeze device.

With the recycle stream occluded and the surgical cooling solution 130 path unobstructed, the surgical cooling solution 130 flows out of the applicator 106 through the end effector 111 and the trocar 110. The end effector includes a tube, such as a stainless steel tube, by way of example, that can be inserted into the trocar 110. As is standard of laparoscopic instruments, the tube is 35 cm (13.8 in.) long and has an outer diameter of 10 mm (0.38 in.). The tip of tube 218 having a nozzle is beveled at a 30° angle. To prevent damaging the kidney, the tip of the nozzle will be smoothened so that there are no sharp points. A recycle stream through the recycle transport system 104B or recyle line provides for a steady stream of fresh slush coming to the applicator 106. In some embodiments the slush flow cycles, in some embodiments, continuously to the applicator 106 and back to the hopper 118 of the surgical cooling solution generator 102 until the surgeon operates the control mechanism which opens a pathway down the end of the applicator 106 and into the patient's body 107. This eliminates the need to catch and store the run-off from a continuously flowing slush stream and keeps the line primed so that in the ‘off’ position slush in the applicator and in the length of tubing (which are both exposed to room temperature) does not melt.

Another implementation of the invention is a method for providing a cooling solution to a body cavity during surgery. The method includes transmitting a continuous flow of the cooling solution to an applicator, selectively delivering a portion of the continuous cooling solution flow to the body cavity, and recycling the non-delivered portion of the cooling solution to the cooling solution generator. The method can include the process of generating a cooling solution having a predetermined consistency. In the method in which a cooling solution is generated, the cooling solution can be further filtered to remove particles greater than a predetermined dimension. This process can help to provide the desired or predetermined toothpaste-like consistency from an aqueous working solution having a 0.9 percent saline concentration. The generation of the cooling solution can further include the step of grinding the cooling solution to reduce the size of the large particles to less than or equal to a predetermined size. The method can also include pressurizing the continuous cooling solution flow. In operation, the system 100 allows continuous adjustments of the flow and delivery of the cooling solution 101 to the organ. The magnitude of pumping pressure to produce a specific flow rate of the cooling solution 101 is a complex function of the hydraulic resistance of the transport system 104 (such as a tubing network) and therefore the viscous properties of the cooling solution 101. Since the cooling solution 101 is not a simple homogeneous fluid, the relationship between the pumping pressure and the flow rate should be determined empirically.

In one embodiment, a roller pump 117 generates pressures up to 2.07×10⁵ N/m² (30 psi), which is estimated to be sufficient for pumping the slush. In operation, adjustments can be made to the system to adjust the amount of the cooling solution 101 (such as an ice slush) applied to the organ.

Another factor to consider is the desired cooling effect of the cooling solution on the applied body part, organ or tissue. For example, an approximate estimate of the amount of energy to be absorbed from an entire kidney by the cooling medium can be made using a simplified heat-transfer analysis. This requires knowledge of the typical organ dimensions and thermal properties. One such example is provided in the following table:

TABLE 1
Kidney Properties

| PROPERTY | VALUE | UNITS |
|---|---|---|
| MASS (M_{K}) | 150 | G |
| THICKNESS (L) | 3 | Cm |
| DENSITY (P) | 1.05 × 10³ | kg/m³ |
| SPECIFIC HEAT (C_{P}) | 3.9 | kJ/kgK |
| DIFFUSIVITY (A) | 1.3 × 10⁻⁷ | m²/sec |

The following equation [1] yields that amount of energy (heat) that must be removed from the kidney to cool the organ from 37° C. to 15° C.:
Q=ΔU=mₖ└u_{f}-uᵢ┘=mCΔT=(0.15 kg)(3.9 kJ/kgK)(15-37K)=−12.87 kJ [1]

Thus, 12.87 kJ of thermal energy should be absorbed by the cooling solution 101. The quantity (mass) of the ice slush cooling solution 101 that must be melted to absorb this amount of energy is determined by equation [2]:
mᵢh_{sf}=12.87 kJ [2]
Hence, based on a heat fusion of 335 KJ/kg is provided by equation [3];

$${m}_{i} = \frac{12.87 kJ}{{h}_{sf}} = \frac{12.87 kJ}{335 \frac{kJ}{kg}} = 0.039 kg = 39 g \qquad [ 3 ]$$

Therefore, 39 grams of the ice slush cooling solution 101 needs to be melted completely to absorb the necessary energy from the kidney. Next, the time required for the core temperature of the kidney to drop from its initial value of 37° C. to 15° C. is estimated conservatively from a one-dimensional transient analysis of a slab of tissue, one surface of which is cooled to 0° C. at time zero. The analysis shows that the temperature of tissue 3 cm deep (distance from the ice layer) will reach 15° C. in a maximum of 40 minutes.

In one embodiment, a cooling solution system and method cools an organ, such as a kidney uniformly to 15-25° C. by applying slush to the top surface of the organ, such as the cortex of the kidney. Some embodiments of the invention apply slush to cool all renal tissue to this range. The flows through a surgical interface, an end effector instrument or applicator can have standard laparoscopic tool dimensions (35 cm in length and maximum of 10 mm in diameter). The surgeon controls the flow rate of the slush easily and conveniently, and the consistency of the slush can be modulated. Some embodiments of the invention provide for minimal operator control and intervention to ensure slush consistency and priming of the slush to the applicator.

In one embodiment, six subject animals were evaluated with an open group and a slush group. In all subject cases, the subject kidneys were successfully cooled to a temperature of 15-25° C. within 10 min and were maintained at the target temperature for 1 hour. Core body temperature for the slush group was decreased by 3 degrees Celsius, but did not change in the open group. Renal (tissue around and related to the kidney) temperatures for the slush group quickly returned to normal upon unclamping of the renal hilum (within 10 min) but the open group required up to 30 min to reach baseline temperature.

In one tested embodiment, a surgical cooling solution generator generated 13 L of slush in one hopper in 3 hours, and 6 L in both hoppers in 1.5 hours. In these cases, a readily-available 0.9% saline (9 g NaCI/L deionized water) slush solution was prepared. The time for preparation was adjusted to ensure the proper consistency of the resulting cooling solution. One embodiment of the system 100 provided a continuous flow of the cooling solution 101 through the system 100 thereby preventing melting and the cooling solution 101 jams that tend to occur if the cooling solution 101 is halted for more than a few seconds. As the system 100 continued to pump during the entire surgery, no the cooling solution 101 jams, plugs, or pressure buildup occurred.

During the tests, five kidney temperature probes were placed at different depths in the kidney to document temperatures in the cortex (superficial portion) and medulla (deep portion). The probes measured local temperatures within the kidney. The ice slush cooling solution 101 was blanketed over the kidney. The probe and body temperatures were recorded every 10 minutes.

To reduce the melt water run-off in the subject, all of the unfrozen water in the cooling solution generator hopper was pumped off before the surgeon applied the slush to the kidney. These measures resulted in keeping the core temperature of the subjects above 35° C.

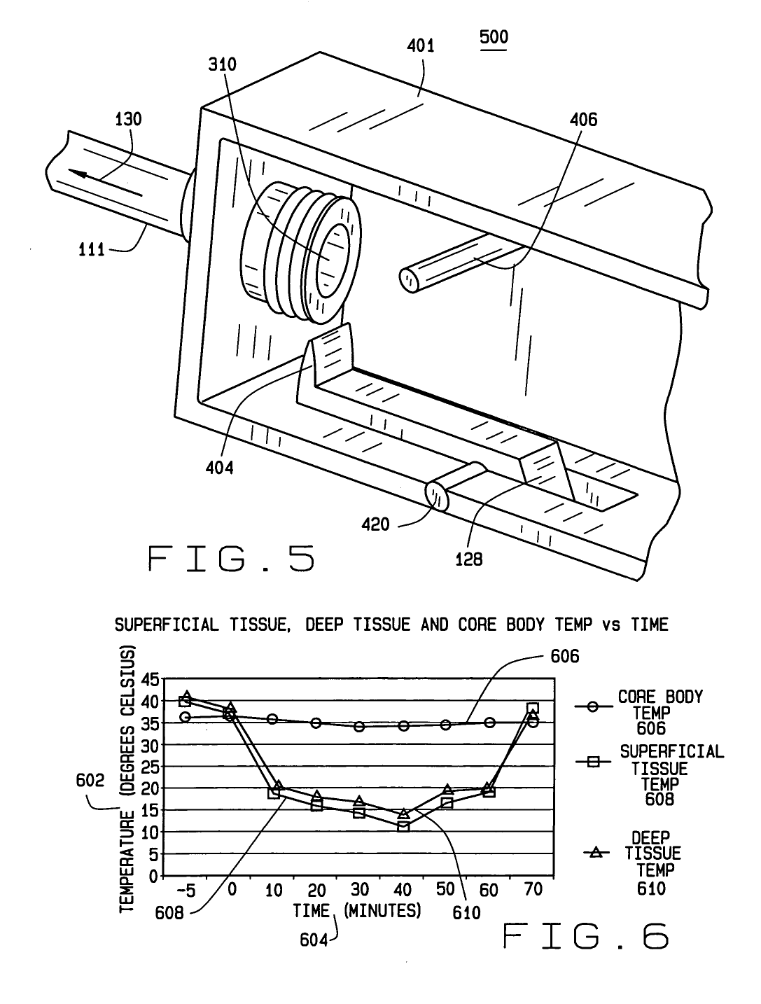

Test results obtained with one exemplary embodiment of the invention are shown in the graph of FIG. 6. The vertical axis 602 is the measured temperature in degrees Celsius and horizontal axis 604 is time in minutes. Three points were measured on the patient during the test surgery. The core body temperature of the test patient is shown by graph line 606. The temperature of the superficial tissue in the body cavity and about the organ is shown by graph line 608. The temperature of the deep tissue within the organ being cooled is shown by graph line 610. As shown in FIG. 6, the application of the cooling solution 101 resulted in a rapid cooling, less than ten minutes, of both the superficial tissue (graph line 608) and the deep tissue (graph line 610), with only a slight decrease in the core body temperature (as shown by line 606) occurring.

The tests of another embodiment demonstrated cooling a kidney to 15-25° C. for over one hour. After the renal artery was unclamped the tissue temperature rose to above 25° C.

During use, in order to minimize associated systemic hypothermia that can result from the overflow of the melted slush to other organs, a trained anesthesiologist can monitor the patient's core temperature and keep the patient warm during the procedure.

As such, some embodiments of the invention provide medical personnel the opportunity for more time to perform surgical procedures and provide for reduced negative effects to patients following the surgery. Additionally, when using the system with an end effector, some embodiments of the invention will increase the opportunity for laparoscopic partial nephrectomy operations thereby making less invasive surgery an option for many other types of surgery.

As with any surgical system, the system or components should be sterilizable for use in numerous applications or disposable. Sterilization can be accomplished by either autoclave, which reaches temperatures of 134° C. (273 F), or by ethylene oxide (EtO) gas sterilization, which reaches temperatures of 60° C. (140 F).

The device is generally described herein to provide improved cold ischemia during laparoscopic partial nephrectomy and laparoscopic donor nephrectomy. Additionally, some embodiments of the system and method have further application for open and laparoscopic surgery, and will improve the standard of care for both due to the cooling properties and capabilities of the cooling solution generation and delivery system and method described herein.

When describing elements or features of the present invention or embodiments thereof, the articles “a”, “an”, “the”, and “said” are intended to mean that there are one or more of the elements or features. The terms “comprising”, “including”, and “having” are intended to be inclusive and mean that there can be additional elements or features beyond those specifically described.

Those skilled in the art will recognize that various changes can be made to the exemplary embodiments and implementations described above without departing from the scope of the invention. Accordingly, all matter contained in the above description or shown in the accompanying drawings should be interpreted as illustrative and not in a limiting sense. It is further to be understood that the steps described herein are not to be construed as necessarily requiring their performance in the particular order discussed or illustrated. It is also to be understood that additional or alternative steps can be employed.

What is claimed is:

1. A system for cooling a body part during surgery, the system comprising:
a surgical cooling solution generator for producing a surgical cooling solution from a solution, said surgical cooling solution having substantial consistency;
a surgical cooling solution transport system for transporting the surgical cooling solution, the transport system including a supply line and a recycle line;
an applicator including a surgical delivery interface and configured for receiving the surgical cooling solution from the supply line, the applicator having a first mode and a second mode, the first mode for routing the surgical cooling solution to the recycle line for recycling of the surgical cooling solution and the second mode for routing the ice slush to a surgical delivery interface.

2. The system of claim 1 wherein the solution is a saline solution having about a 0.9 percent saline concentration.

3. The system of claim 1 wherein the surgical cooling solution generator includes a screening device configured for removing particles of surgical cooling solution having a dimension greater than a predetermined dimension.

4. The system of claim 1 wherein the surgical cooling solution generator is configured for producing surgical cooling solution having a toothpaste-like consistency.

5. The system of claim 1 wherein the surgical cooling solution generator includes a cooling hopper configured for freezing a volume of the solution into the surgical cooling solution.

6. The system of claim 1 wherein the surgical cooling solution generator includes a recycle port for receiving a surgical cooling solution to be recycled.

7. The system of claim 1 wherein the surgical cooling solution generator includes a rotating auger for continuously agitating the surgical cooling solution held within a hopper of the generator.

8. The system of claim 1 wherein the surgical cooling solution generator includes a first rotating auger and a second counter-rotating auger, the first and second augers continuously agitating the surgical cooling solution held within a hopper of the generator.

9. The system of claim 1, wherein the surgical cooling solution generator includes a hopper, a first rotating auger, a second rotating auger, and a grinding device, the first auger configured for continuously agitating the surgical cooling solution held within the hopper and the second auger generating a surgical cooling solution flow through the grinding device.

10. The system of claim 1 wherein the surgical cooling solution generator includes a hopper, a first rotating auger and a second rotating auger, the first auger continuously agitating the surgical cooling solution held within the hopper and the second auger providing an extruding force to the surgical cooling solution transport system.

11. The system of claim 1, further comprising a pump for providing a predetermined pressure within the surgical cooling solution transport system.

12. The system of claim 12 wherein the pump is non-invasive to the transport system and the surgical cooling solution and the pump provides a consistent pressure to the surgical cooling solution within the transport system.

13. The system of claim 1 wherein the applicator includes a handle operated by a user, said handle controlling the selection of the first mode and the second mode.

14. The system of claim 1 wherein the applicator includes variable routing modes providing a plurality of variable combinations of the first mode and the second mode, said variable routing modes providing variable routing of surgical cooling solution to the surgical delivery interface.

15. The system of claim 1 wherein the surgical delivery interface includes an end-effector configured for operation with a laparoscopic trocar.

16. An applicator for dispensing a surgical cooling solution during surgery, the applicator comprising:
an intake port for receiving the surgical cooling solution;
a recycle port for transmitting a portion of the surgical cooling solution for recycling;
a surgical delivery interface for transmitting a portion of the surgical cooling solution to a body cavity; and
a flow control system for controlling a flow of the received surgical cooling solution, the flow control system having a first mode for routing the surgical cooling solution to the recycle port and a second mode for routing the surgical cooling solution to the surgical delivery interface.

17. The applicator of claim 18 wherein the flow control system includes a variable mode for routing a user-defined portion of the surgical cooling solution to the surgical delivery interface.

18. The applicator of claim 18 wherein the flow control system includes a user control for selecting the first mode and the second mode.

19. The applicator of claim 18 wherein the user control includes a handle operated by a user and includes a variable mode for routing a user-defined portion of the surgical cooling solution to the surgical delivery interface, said handle controlling the selection of the first mode, the second mode, and the user-defined portion of the surgical cooling solution.

20. The applicator of claim 18 wherein the flow control system includes at least one of a flow control valve and a tube pinch device.

21. The applicator of claim 18 wherein the surgical delivery interface includes an end-effector configured for operation with a laparoscopic trocar and configured to deliver the surgical cooling solution into a body cavity during the surgery.

22. A method of providing a surgical cooling solution to a body cavity during surgery, the method comprising:
generating a surgical cooling solution having a predetermined consistency from a solution;
transmitting a continuous flow of the surgical cooling solution to an applicator;
selectively delivering a portion of the continuous surgical cooling solution flow to the body cavity; and
recycling the non-delivered portion of the continuous surgical cooling solution flow for inclusion in said transmitted continuous flow of the surgical cooling solution to the applicator.

23. The method of claim 24 wherein generating includes screening the surgical cooling solution to remove particles having a dimension greater than a predetermined dimension.

24. The method of claim 24 wherein the predetermined consistency includes a surgical cooling solution having a toothpaste-like consistency.

25. The method of claim 24 wherein the solution is a saline solution having about a 0.9 percent saline concentration.

26. The method of claim 24 wherein transmitting a continuous surgical cooling solution flow includes pressurizing the transmitted continuous surgical cooling solution flow.

27. The method of claim 24 wherein generating a surgical cooling solution having a predetermined consistency includes grinding the surgical cooling solution to reduce a size of the surgical cooling solution particles to less than or equal to a predetermined size.

28. A method of cooling tissue during laparoscopic surgery, the method comprising:
transmitting a continuous flow of surgical cooling solution to a laparoscopic surgical cooling solution applicator;
selectively delivering a portion of the continuous surgical cooling solution flow to a body cavity through a trocar; and
recycling the non-delivered portion of the continuous flow of the surgical cooling solution for inclusion in said continuous flow of the surgical cooling solution to the laparoscopic applicator.

29. The method of claim 30 wherein the surgical cooling solution includes a 0.9 percent saline concentration.

30. The method of claim 30 wherein the continuous flow of the surgical cooling solution includes a surgical cooling solution having a predetermined consistency of toothpaste.

31. The method of claim 29 wherein transmitting the continuous surgical cooling solution flow includes pressurizing the transmitted continuous surgical cooling solution flow.

32. The method of claim 29 wherein selectively delivering the surgical cooling solution flow includes receiving a user input selecting the portion of the surgical cooling solution flow to be delivered to the body cavity and the non-delivered portion to be recycled.

33. The method of claim 29, further comprising generating a surgical cooling solution having a generally consistent predetermined consistency.
